(19) United States
(12) Patent Application Publication
(10) Pub. No.: US 2010/0217729 A1
(43) Pub. Date: Aug. 26, 2010

(54) SHAVER DISINFECTION SYSTEM FOR EACH CUSTOMER TYPE

(76) Inventor: Yuusaku Takahashi, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 12/738,858

(22) PCT Filed: Nov. 17, 2008
(86) PCT No.: PCT/JP2008/071185

§ 371 Date: Apr. 20, 2010

(30) Foreign Application Priority Data
Nov. 5, 2007 (JP) 2007-311556

(51) Int. Cl. G06Q 90/00; A61L 2/04

(52) U.S. Cl. 705/500; 422/307

(57) ABSTRACT

The technical problem to be solved by the present invention is to develop an insurance system against shaving wounds which makes it obligation for a barber shop to issue a shaver disinfection contents report for proving the sterilizing temperature and sterilizing time of the shaper, the temperature that the shaver reaches, and so on performed by the barber shop in order to ensure execution of shaver disinfection on individual customer basis by the barber shop, and institutionalizes a charging shaver disinfection system which compensates financial burden of the barber shop, thereby relieving both of clients and barbers from shaving injuries. A main application of the present invention is to sterilize the shaver to prevent the customers from being infected with AIDS, hepatitis B, hepatitis C, and other virus caused by the saving wound.

### TECHNICAL FIELD

The present invention relates to a realization of a charging shaver disinfection system in which a barber shop or a beauty salon sterilizes a shaver in front of a client, and performs haircut on individual customer basis using “the sterilized shaver”, and to solve the following technical problem items.

I. There is a case where a used shaver is reused in an altogether disinfection system

I1. To develop a method of preventing “reuse as is” of the used shaver
I1(1). To develop a customer-present execution and explanation system for shaver disinfection

II. There is no system to prove that virus is sterilized

II1. To fix a scheme to apply a method of electric signal measurement and analysis for a shaver disinfector

II2. To develop a shaver disinfection data collecting and accumulating system

III. There is no shaver disinfection contents report issuing system

III1. To develop a shaver disinfection contents report column preparation system

III2. To develop a consensual validation recording column for presence or absence of a shaving wound preparation system

IV. There is no charging shaver disinfection system

IV1. To develop a charging shaver disinfection system

V. There is no insurance system against a shaving wound

V1. To develop an insurance system against shaving wounds caused by a disinfected shaver

### BACKGROUND ART, AND DESCRIPTION OF PROBLEMS

Background Art I. In an altogether disinfecting system, a used shaver may be reused

In most barber shops, shaver disinfection is not performed. However, there are a few barber shops which perform the disinfection of the shavers. In these shops, for example, a method of disinfecting a plurality of the shavers altogether in a large steam boiler, and using the same by taking out therefrom one by one is taken.

A merit of “the altogether disinfection is that so-called “a preparation time” can be reduced. However, a demerit is that large-scaled disinfection equipment is required, and hence consistent shaver disinfection cannot be performed in a narrow shop front area. Therefore, a disinfection process might include a process step of performing part of the process out of sight of a customer, or a process step that the customer cannot count a disinfection time, and hence we cannot say that mixed storage of the disinfected shavers and the used shavers “cannot happen”, and taking the used shaver by mistake instead of the disinfected shaver is within the bounds of possibility.

The fact that “mixing”, that is, a case of using the shaver used for a previous customer and hence is contaminated might occur is a fatal defect, and occurrence of “mixing” is an absolute condition which should be eliminated irrespective of its merits.

Problem I1. To develop a method of preventing “reuse as is” of the used shaver

A method of starting disinfection of “a shaver used for a customer” in his/her sight with explanation when “the exact same customer” is seated on a hair-cut chair, and finishing disinfection of “the shaver used for the exact same customer” before “scissoring work” is ended is a most safe and reliable way for preventing the mixing from the barber side.

From the customer side as well, the mixing and reusing of a contaminated shaver cannot happen only by watching a shaver being disinfected in his/her sight (a customer-present execution and explanation system for the shaver disinfection) and confirming that he or she is shaved by using “the exact same shaver”.

In order to solve the Problem I1, Problem I1(1) shown below must be realized using a compact heating shaver disinfector which is capable of performing a total disinfection process through.

Problem I1(1). To develop the customer-present execution and explanation system for the shaver disinfection

Background Art II. There is no system to prove that virus is sterilized

A social unrest is increased by the fact that we cannot bring a shaver disinfection inexecution practice, which is a law violation, into action.

The customer side must establish a fair lawsuit environment for the barber shop which does not perform the shaver disinfection.

The shaving work is associated with a risk of causing an incised wound at high probabilities. At that time, if the disinfected shaver is used, it may be treated with a simple after care. However, if there is a doubt that a shaver attached with intractable disease-causing agents such as HIV, virus for Hepatitis B or C is used, it is litigable and the customer must file a lawsuit.

The shaver disinfection in shop front areas of barber shops in status quo is unexecuted or insufficient in many cases. However, the barber shops do not present any evidence relating to the disinfection to the customer. An only solution to solve this problem is to present a rational record of execution of sufficient sterilization in a shaver sterilizing process to the customer.

Means II1. To fix a scheme to apply a method of electric signal measurement and analysis for a shaver disinfector

Since there is no method of proving virus sterilization so far, there is a serious credibility gap between the barber shops which make no attempt to perform the barber disinfection and customers focusing on infection with fatal diseases.

The reason resides eventually in the fact that there is no method of expressing the shaver disinfection quality quantitatively. However, a method of measuring a voltage and a current flowing in respective parts of the shaver disinfector and converting the same into temperature data (the method of electric signal measurement and analysis for the shaver disinfector) individually on individual customer basis found by the present inventor enables quantification and visualization of the disinfection quality and brighten the prospects of guaranteeing the shaver disinfection quality.

According to the method of electric signal measurement and analysis for the shaver disinfector, not only the temperatures of the parts which constitute the shaver disinfector, but also the temperature of a heating chamber, the temperature of the shaver, and a heating time are acquired as data and can be printed on a paper. Therefore, it can be presented to the customer as “the shaver disinfection contents report” (this will be described in Means III) and used as “documentary evidence of performance of the shaver disinfection”.

To develop the virus sterilization certifying system is to issue the shaver disinfection contents report. In order to do so, it is necessary to develops Means II2. A shaver disinfection data collecting and accumulating system.

Means II2. To develop the shaver disinfection data collecting and accumulating system

In order to achieve Means II2, the following problems must be realized.

Problem II2(1). Acquisition of electric signals from the shaver disinfector

Problem II2(2). Transmission and accumulating the acquired electric signals

Problem II2(3). Attachment of voice guide equipment or the like to the shaver disinfector

The issue of the shaver disinfection contents report is achieved by performing Means III1, III2 described in Background Art III using the systems of Means II1 and Means II2.

Background Art III. There is no shaver disinfection contents report issuing system

By utilizing the method of electric signal measurement and analysis for the shaver disinfector, the documentary evidence which can be used for the lawsuit can be prepared and issued by combining data of the accurate temperatures collected from the respective parts of the disinfector and the shaver.
A system which allows third parties to rationally determine whether the disinfection contents are adequate or not can be arranged on the side of the barber shops by preparing the shaver disinfection contents report including temperature data of the heating disinfection, temperature data that the shaver reaches, data on duration of heating, or the like in correct execution of the disinfection of the shaver printed without modification on individual customer basis by using the disinfector which can sterilize virus.

While explaining the execution of the disinfection in customer's sight, the temperature that the shaver reaches is “actually presented” by a liquid crystal temperature display of the shaver disinfector, whereby the record is used not only for setting the customer's fears about the shaver disinfection quality at rest, but also as evidence of “actual execution of the legally settled shaver disinfection on individual customer basis” by printing and handing over the same to the customer as the shaver disinfection contents report. Therefore, the shaver disinfector requires proper maintenances and regular inspections by the third party.

It is recommended to provide two columns, namely, a shaver disinfection contents report column and a consensual validation recording column for presence or absence of shaving wounds in the shaver disinfection contents report.

The reason is that the shaver disinfection contents report is “a certificate of application of a treatment to avoid infection with intractable diseases on the shaver”, but several novel businesses as follows can be born:

1. By regarding this report as “a guarantee of not being infected with the intractable diseases” and getting in a shaver disinfection fee from the customer in exchange, a business to collect the cost of equipment required for the installation of this system can be managed without depending on the government or the banks.
2. Granted that a shaving wound is made, this report can be attached when application of the insurance for medical treatment is issued as the evidence that they are a simple wound irrelevant to virus.

Problem III1. To develop a shaver disinfection contents report column preparation system

Problem III2. To develop a consensual validation recording column for presence or absence of shaving wounds preparation system

Background Art IV. There is no charging shaver disinfection system

For the moment, there is neither a bounty system nor a tax reduction system for introduction of a new shaver disinfection technology in Japan, and several years will be required for passing a new law and institutionalizing these systems. Since the banks will hesitate to positively accommodate small-scale barber shops with loans, it will take significant time until the shaver disinfection system on individual customer basis becomes popular. If we imagine that there will be increased number of new patients who are injected with or present the symptoms of AIDS or hepatitis, a mechanism which allows the barber shops to collect funds immediately for capital investment for the disinfection equipment by their own ability is seriously desired.

The customers will pay for a haircut without risk of virus infection if the sterilization is reliable, and the barber shops will be able to purchase the shaver disinfection system on individual customer basis by saving the received shaver disinfection fees, so that the desires and the needs of both sides will be met.

After having cleared up the capital investment for the disinfection equipment, the shaver disinfection fees from then on will support the management of the barber shops as increment of the benefit.

I believe that prompt realization of such system that the barber distributes the certificates of virus sterilized, which are realized by the Problems III1 and III2, to all customers and receiving the shaver disinfection fees from the customers (=the charging shaver disinfection system) in exchange is an ideal political measure.

Problem IV1. To develop the charging shaver disinfection system

Background Art V. There is no insurance system against shaving wounds

Although a shaving wound may be made very often as an actual problem, the barber shops do not give medical treatment except for applying simple vulnerary, and are not insured. In the barber shops using the shaver disinfector which is able to sterilizing virus, even when the shaving wound is made, as it is “a simple shaving wound which is irrelevant to the intractable disease”, the barber shops can continue business in piece if there is a small sum insurance system against the shaving wound.

In order to realize Background Art V, a technology of preparation of items III1 and III2 in Problem III described in Background Art III is necessary as a premise, and then Problem V can be realized.

Background Art III. To develop a shaver disinfection contents report issuing system

Problem III1. To develop a shaver disinfection contents report column preparation system

Problem III2. To develop a consensual validation recording column for presence or absence of shaving wounds preparation system

Problem V1. To built an insurance system against shaving wounds caused by a disinfected shaver

Description of the state of the background art which hinders realization of the shaver disinfection on individual customer basis and various problems for executing and establishing the shaver disinfection on individual customer basis will be ended here. Subsequently, in a paragraph of “Disclosure of the Invention”, analysis and inspection executed by the inventor in the order of these problems, and measures for realizing these problems will be described.

### DISCLOSURE OF INVENTION

### (Reference) Structure and Performance of Radiating Shaver Disinfector

A shaver disinfector used in the present invention is of a system which sterilizes virus and microscopic organisms attached to a surface of a shaver by heating the shaver, and includes “a system of heating the shaver put in a water trough” or “a system of heating the shaver in the air directly by radiant heating from a heat source”. The structure and the performance of the radiating shaver disinfector in the applied system are generally as follows.

As shown in FIG. 1, two heaters each formed of a mica plate 3 with a heating element ribbon wound therearound are connected in series, and are assembled into one heating panel using mica plate assembly fittings 1 and 5. As shown in FIG. 4:

(1) the two heating plates 3 and 3 are mounted to a frame 20 so as to oppose to each other,
(2) holders 18, 19, 18 for holding a shaver 17 with a cutting edge faced upward are mounted on the frame 20 so as to be positioned at a center between the two heating plates in (1), and fixed so that the distances between a centerline of the shaver and heating surfaces of the heating plates on the outer sides thereof are the same on both left and right sides as a result of (1) and (2).

When performing the heating disinfection with the shaver inserted in the holder, the radiating shaver disinfector has a function to increase the temperature of the shaver to a temperature around 160° C. within two minutes after having started the heating, activate a temperature adjusting function to lower the temperature once to a lower limit temperature around 120° C., and start to increase the temperature secularly by degrees while moving slightly up and down, then shift to declination around an upper limit temperature around 140° C., and keep the temperature of the shaver stably at a temperature around 130° C.

By setting the heating time to a period from 2 minutes to 5 minutes inclusive, the virus or the like dies out.

When the radiant heating is performed from close range at a heat source temperature from 1000° C. to 800° C., the temperature of the virus on the side of the heat source with respect to the shaver is increased before the shaver as a matter of course. On the other side, when the shaver is heated, the shaver heats up virus bodies from below the virus with transferred heat. Protein in the interior of the virus is heated to a temperature not lower than 60° C. even with the transferred heat from the shaver at 130° C., and is transformed into denatured protein, which causes the virus to die out.

In addition, radiation of high temperature near 1000° C. from the close range can not help burning out the virus which needs moisture in the periphery thereof from inside and outside.

The present inventor asked Adjunct Professor Nakashima (as of August, 1995) in Department of Microbiology, Yamanashi Medical Collage to measure an HIV inactivation effect of the present disinfector, and as a result, sterilization of HIV virus is successfully achieved within a very short time (one minute).

The disclosure of the invention shown below will be sorted out with Roman Figures (Capitals) as “Invention Numbers” shown on the left ends of Background Art and will be described thereby.

“Disclosure of Invention Number I. Development of Customer-Present Execution and Explanation System for Shaver Disinfection”

Confirmation, inspection, and study performed in order to solve this problem will be as follows.

I1(1)[1]. Object of Customer-Present Execution and Explanation System for Shaver Disinfection

The object of this system is to explain the effect of the shaver disinfection on sterilization and the fact that there is no probability of infection with the intractable disease with the sterilized shaver even when a shaving wound is made, show and give a customer a picture of a process step of the shaver disinfection, alleviate the anxiety of the customer, and convince the customer that he or she loses nothing by paying the fee for the shaver disinfection.

Therefore, it should be an explaining system having a mechanism that allows the customers (even children) to confirm that the disinfection is executed reliably for them.

I1(1)[2]. Execution Procedure of Voice Guide according to Haircut Process Step

A barber puts the customer in a haircut chair and covers a sheet cover for covering the clothing and, immediately after that, inserts the shaver 17 into the shaver holders 18, 19, 18 of the shaver disinfector shown in FIG. 4, and presses a push-button switch 12 shown in FIG. 3.
When the shaver disinfector is activated thereby, an announcement of a voice guide starts immediately, and an explanation corresponding to the disinfection process step of the shaver disinfector is given.

The barber performs the shaver disinfection using the shaver disinfector in the customer's sight while using scissors. A voice guide system explains the method and effects of the shaver disinfection in parallel while the shaver disinfection is in progress.

During a scissoring work, the progress state of the disinfection should be presented to the customer totally “from the beginning to the end” while explaining to the customer the fact that the disinfection is properly performed in the shaver disinfector.
When viewed from a different standpoint, the customer is also bound in duty to confirm the fact that it is properly disinfected.

I1(1)[2]i. Start of Heating

When the barber presses the push-button switch (red) 12 shown in FIG. 3, a buzzer sounds, a neon lamp (red) 11 is illuminated, and the heating plates 3 in FIG. 4 and a timer are activated.

At this time, the voice guide system is activated and the following announcement is given.

Beep, the shaver disinfection is performed at a high temperature from now for n minutes.

Note: a figure authorized by Ministry of Health is used as the heating time n minutes.

After having activated the shaver disinfector, the barber starts the scissoring work immediately.

Note: start heating by means other than the push-button switch.

It is also possible to acquire an electric signal indicating start heating by a detection switch such as a limit switch or a photoelectric switch which senses the insertion of the shaver and connect the same to the buzzer or the display lamp instead of the push-button switch. The detection switch such as the photoelectric switch may be suitable for the shaver disinfector of a boiling-type shaver heating system in which a cutting edge of the shaver is soaked into a boiling water trough and a handle is bent and hooked on an edge of the trough of the boiling water. As a matter of course, starting by the push-button switch is applicable without problem.

I1(1)[2]ii. End of Heating

When the heating time entered to the timer in advance is elapsed, a power distribution circuit to the heating plates is opened, the buzzer sounds, a neon lamp (green) 10 is illuminated, and the neon lamp (red) 11 is extinguished. At this time, the voice guide system is activated and the following announcement is given.

Beep, the high-temperature shaver sterilization is completed now. AIDS virus, hepatitis B virus, hepatitis C virus, etc. are died out from the shaver. As the disinfected shaver is used for your haircut, even when a shaving wound is made, rest assured that you will not be infected with AIDS or hepatitis.

Therefore, the shaving wound which may be made in this shop can be treated with a simple medical attention. The shaver is cooled down from now.

I1(1)[2]iii. Start of Cooling

At the same time with the announcement of I1(1)[2]ii, a fan 22 for cooling the heated shaver down starts to turn.

I1(1)[2]iv. End of Cooling

When the cooling time entered to the timer in advance is elapsed, a power distribution circuit to the cooling fan is opened, the buzzer sounds, the fan 22 is stopped, and the neon lamp (green) 10 is extinguished.

At this time, the voice guide system is activated and the following announcement is given.

Beep, cooling down of the shaver is completed now. Now, your face is shaved with the disinfected shaver.

When the haircut is ended, a shaver disinfection contents report will be issued. Please receive and keep it.

If a shaving wound is found, confirm the wound with the barber and receive the signature of the barber.

With the voice guide according to the process steps of the shaver disinfection as described above, the customer is able to know that the shaver is actually disinfected, the virus of intractable diseases is sterilized, even when a shaving wound is made, the wound does not cause infection with AIDS, hepatitis, or the like, the wound can be treated with a simple medical attention like an injection scar, and so on, has a feeling of safe and enhances understanding of a charging shaver disinfection system.

I1(1)[2]v. Haircut Process Step from then on

The barber pulls out the shaver from the shaver disinfector after having finished the cooling, and places the shaver on a table in front of the customer.

From then on, the barber continues a scissoring process or uses the shaver to start a shaving process. If a shaving wound is made in the shaving process, the barber must write a general description of the shaving wound in “a consensual validation recording column for presence or absence of shaving wounds” in shaver disinfection contents report and ask the customer to confirm the written contents.

Consequently, the customer and the barber sign the shaver disinfection contents report with each other and keep one each copy.

The barber must take a procedure for application of the insurance against wounds according to the degree of the wound depending on the customer's wish.
A push-button switch (black) 9 on an upper surface of a control board housing of the shaver disinfector is for emergency stop.

“Advantageous effects of Invention Number I. Customer-Present Execution and Explanation System for Shaver Disinfection in relevant to the related art”

For the moment, the shaver disinfector employing the customer-present execution and explanation system for the shaver disinfection does not exist in the market. However, the customer can have a feeling of safe by viewing the shaver disinfection performed reliably in a correct process step by the shaver disinfector having the present system mounted thereon.

“Best Mode for Carrying Out Invention Number I. Customer-Present Execution and Explanation System for Shaver Disinfection”

Carrying out in combination with the radiating shaver disinfector which is configured to achieve the sterilization in a short time described at the outset (reference) in DISCLOSURE OF INVENTION is the best mode.

“Embodiment of Invention Number I. Customer-Present Execution and Explanation System for Shaver Disinfection”

Embodiment is now in the course of preparation and hence is not given here.

“Disclosure of Invention Number II. To Develop System to Prove that Virus is Sterilized”

As described in the paragraph of Means II1. To fix a scheme to apply a method of electric signal measurement and analysis for a shaver disinfector in TECHNICAL FIELD, the method of electric signal measurement and analysis for the shaver disinfector is a system to collect electric signals issued from various parts of the shaver disinfector and an electric signal issued from a temperature sensor attached to the shaver disinfector as shaver disinfection data, process and analyze the shaver disinfection data with a personal computer, print the temperature that the shaver reaches and the length of the heating time and output as a certificate of the disinfection quality.

Therefore, development of the system to prove that virus is sterilized can be said to be equal to development of a shaver disinfection data collecting and accumulating system using Means II1. A scheme (hardware environment) to apply a method of electric signal measurement and analysis for a shaver disinfector.

What were confirmed, inspected, and studied for Invention Number II2. To develop the shaver disinfection data collecting and accumulating system are as follows.

The shaver disinfection data collecting and accumulating system acquires an electric signal indicating start of heating or start of cooling of the shaver performed by the shaver disinfector and electric signals relating to the disinfection temperature and the temperature of the shaver to be disinfected sensed by a sensor of a temperature adjusting circuit of the shaver disinfector, other temperature sensor or the like, and processes these electric signals into disinfection data such as a disinfection starting time, a disinfection ending time, the length of a disinfection time, and so on. In addition, data such as the date, month, and year of execution of the disinfection, the name and a photo of the customer, the name and a photo of the barber or the hair designer are processed in combination into shaver disinfection database.

On the other hand, a management information processing system such as sales, accounting is developed by acquiring data such as a received haircut fee and a received shaver disinfection fee from database of a different system and combining the same with the above-described disinfection data.

Activation and stop of the shaver disinfection data collecting and accumulating system are operated in conjunction with activation and stop of the shaver disinfector, and the shaver disinfection data should not be collected and accumulated with other source or method.

The shaver disinfection data should be acquired only from the shaver disinfector, and a scheme to prevent the interested party from correcting or modifying later without authorization is inevitable.

Therefore, when designing the present system, it is necessary to integrate an interlock system to prevent a person other than a registered authorized person from accessing the database and changing the setting of disinfection conditions.

Hereinafter, a method of solving and developing the following three problems for developing Problem II2 will be described.

Problem II2(1). Acquiring of electric signals from the shaver disinfector

Problem II2(2). Sending and accumulating the acquired electric signals

Problem II2(3). Attachment of voice guide equipment or the like to the shaver disinfector

II2(1). Acquiring of the electric signal from the shaver disinfector

First of all, how to acquire electric signals indicating the start of heating, the end of heating, the start of cooling, the end of cooling, the temperature of the heating chamber, the water temperature in a heating trough, and the temperature of the shaver to be disinfected, which are the most important data for proving whether it is disinfected or not is a question.
There are several methods of acquiring the electric signals as shown below.

The electric signals indicating the start of heating, the end of heating, the start of cooling, and the end of cooling are taken out from power cable of the heater and the fan, are connected to a terminal or a terminal base, a USB 2.0 port connector or a wireless USB 2.0 port connector provided on the shaver disinfector after having adjusted voltage and current values to match information communication equipment and environment.

Control signals for the heater and the fan are acquired from a microcomputer which forms a control circuit of the shaver disinfector, and are connected to the terminal or the terminal base, the USB 2.0 port connector or the wireless USB 2.0 port connector provided on the shaver disinfector.

By using a contact point of a relay which forms the control circuit of the shaver disinfector as is, the contact point current and voltage values are connected to the terminal or the terminal base, the USB 2.0 port connector or the wireless USB 2.0 port connector provided on the shaver disinfector after having adjusted the same to match the information communication equipment and environment if necessary.

The electric signals relating to the temperatures such as the temperature of the heating chamber, the water temperature in the heating trough are acquired from a sensor circuit of a temperature adjusting mechanism of the shaver disinfector and are connected to the terminal or the terminal base, the USB 2.0 port connector or the wireless USB 2.0 port connector provided on the shaver disinfector after having adjusted the voltage and current values to match the information communication equipment and environment if necessary.

A sensor for measuring the temperature other than the sensor circuit of the temperature adjusting mechanism such as the temperature of the shaver is configured by attaching a platinum temperature measurement resistor to an adequate position on the shaver holder of the shaver disinfector so as to come into contact with the shaver, integrating various circuits required for sensing the temperatures in a substrate, and storing into the housing for the control board, the push-button, and the confirmation lamp system indicted by reference numeral 8 in FIG. 3. The electric signals adjusted so as to be converted into temperature values in the information communication equipment and environment are acquired from the platinum temperature measurement resister via the substrate of the various circuits required for sensing the temperature, and are connected to the terminal or the terminal base, the USB 2.0 port connector or the wireless USB 2.0 port connector provided on the shaver disinfector with a cable.

Although it is described that “integrating various circuits required for sensing the temperatures in a substrate” above, a method of linearize a current of the sensor to a degree which allows the usage of a calculation formula to convert the voltage value to the temperature value cannot be described in detail because the designs and combinations are significantly different depending on the part numbers of the sensor or the manufacturers, an expression “various circuits required” is used.

The calculation formula to convert the voltage value adjusted by the various circuits required for sensing the temperature into the temperature value is installed in the computer in advance by a CD sold with the shaver disinfector.

As regards not only the temperature data, but also the electric signals indicating the start of heating, the end of heating, the start of cooling, and the end of cooling as well, the circuits for adjusting the current and voltage values so as to be fed to the information communication equipment and environment are naturally required, and are stored in the housing for the control board, the push-button, and the confirmation lamp system indicted by reference numeral 8 in FIG. 3.

In the small barber shops or beauty salon, data communication or printing using a wireless LAN is specifically required by force of necessity. However, since the wireless LAN between personal computers is realized, if it is necessary to specifically adjust the current and voltage values of the electric signals of the various data of the shaver disinfector for the transmission from the wires USB 2.0 port, corresponding adjusting circuits are also stored in the housing for the control board, the push-button, and the confirmation lamp system indicted by reference numeral 8 in FIG. 3.

In the embodiment (experimental machine), there is one sensor for the temperature adjusting circuit, and I took great pains to determine the position to attach the sensor. However, in a practical machine it is desirable to collect a plurality of data such as data on the temperature in the heating chamber, data on the water temperature in the heating trough, and data on the shaver temperature, so that a plurality of temperature sensor specifically for collecting the data are preferably provided.

The data of personal names of the customers who have received the shaver disinfection are accumulated in a customer database in the computer, and are stored as disinfection information data on individual customer basis in association with the above-described disinfection data. If photographs of the customer's faces are required, a personal authentication system such that the customers are photographed by a camera installed upper front of the customer when he or she is seated on the haircut chair is built in the computer.

By employing customer cards in the barber shops or the beauty salons, these processes are achieved easily.

II2(2). Sending and accumulating the acquired electric signals

The electric signals indicating the start of heating, the end of heating, the start of cooling, and the end of cooling of the shaver disinfector, the temperature in the heating chamber, the water temperature in the heating trough, the temperature of the shaver and so on acquired to the terminal or the terminal base, the USB 2.0 port connector or the wireless USB 2.0 port connector provided on the shaver disinfector are transmitted as is to the USB 2.0 port of the computer, and sent to database software or spreadsheet software of the computer, stored and accumulated in MO drive or the like, and constitute the shaver disinfection database.

II2(3). Attachment of voice guide equipment or the like to the shaver disinfector

When notifying and describing the fact of the disinfection to the customers with the voice guide system described in paragraph 1.1, it is effective to use with the buzzers for attracting the customers' attention.
The voice guide equipment may be integrated entirely (for example, a compact tape recorder type) or partly (for example, only a speaker) into the shaver disinfector.

The equipment of the voice guide also requires input and output of the data and, by connecting the same to the information communication equipment and environment such as the computer, the internet, or the printer using the terminal or the terminal base for acquiring and inputting the electric signals, or the USB 2.0 port or the wireless USB 2.0 port provided on the shaver disinfector and organizing and accumulating the transmitting, receiving, and guiding contents text, it can be used with various OS or software in addition to the usage as the voice guidance, so that a guide system superior in sales with the disinfection information of the shaver disinfector as a key focus is realized.

A liquid crystal temperature display which allows the customer to visually recognize whether or not the shaver is heated until temperatures which are able to sterilize the virus actually while listening to the voice guide is also connected to the information communication equipment and environment such as the computer, the internet, or the printer using terminal or the terminal base for acquiring and inputting electric signals, or the USB 2.0 port, or the wireless USB 2.0 port provided on the shaver disinfector, to achieve transmission, reception, and display of the numerical values of the temperatures.

“Advantageous effects of Invention Number II2. Development of Shaver Disinfection Data Collecting and Accumulating System in relevant to the related art” A technology to collect the data on the sterilizing temperatures at which the shaver is disinfected from the shaver disinfector on individual customer basis and a technology to present the data to the customers to let them know the disinfection quality of the barber shops are new technology, and demonstrate advantageous effects in terms of sales in comparison with those in the related art.

“Best Mode for Carrying Out Invention Number II2. Development of Shaver Disinfection Data Collecting and Accumulating System”

Carrying out in combination with the radiating shaver disinfector which is configured to achieve the sterilization in a short time described at the outset (reference) in DISCLOSURE OF INVENTION is the best mode.

“Embodiment of Invention Number 112. Development of Shaver Disinfection Data Collecting and Accumulating System”

Embodiment is now in the course of preparation and hence is not given here.

“Disclosure of Invention Number III. Development of Shaver Disinfection Contents Report Issuing System” Confirmation, inspection, and discussion performed in order to solve this problem will be as follows.

(Significance of the Shaver Disinfection Contents Report)

In Means II, the confirmation, notification, and description of the fact that the shaver disinfection is performed mainly by voice or visually are performed to allow the customers to understand the fact that the corresponding shop has performed the shaver disinfection, the significance, the contents, and the effects thereof. Means III is that the barber prepares detailed contents of the disinfection of the shaver that is used for the corresponding customer as the shaver disinfection contents report, which becomes a written evidential record, when finishing the haircut and hand the same to the customer.

The shaver disinfection contents report is a documentary evidence proving the execution of the shaver disinfection for preventing the virus of the intractable diseases from entering inside the body, and is an effective social system for avoiding occurrence of the lawsuit over the disinfection quality (or to encourage to file a lawsuit for bring a shaver disinfection inexecution practice to a stop) at a later date.

On the other hand, a charging shaver disinfection system can be developed by regarding the shaver disinfection contents report as a guarantee for preventing the infection with the intractable diseases such as AIDS or hepatitis. In addition, even when a shaving wound is made, in case of the shaving wound associated with the shaver disinfection contents report, the wound can be regarded as a simple wound irrelevant to the virus because it is made by the shaver which is disinfected as prescribed, and hence the report can be used as an attachment for the application of the insurance against the wounds, which results in a merit that several types of businesses such as development of the insurance system against shaving wounds can be created.

The shaver disinfection contents report and the shaver disinfection contents certificate have the same meaning and same contents. However, in this application, the term the shaver disinfection contents report is used.

Problem III. To develop a shaver disinfection contents report issuing system

The shaver disinfection contents report is made up of the shaver disinfection contents report column and the consensual validation recording column for presence or absence of shaving wounds.
Therefore, “Problem III. To develop a shaver disinfection contents report issuing system” can be divided into “Problem III1. To develop a shaver disinfection contents report column preparation system” and “Problem III2. To develop a consensual validation recording column for presence or absence of shaving wounds preparation system”.

Problem III1. To develop a shaver disinfection contents report column preparation system

The shaver disinfection contents report column includes required data or items selected from recording data on heating and energization of the shaver disinfector, the data on the temperature in the heating chamber, the data on the water temperature in the heating trough, and data on the shaver temperature, the data on the length of the disinfection time, the date, month, and year of execution of the disinfection, the time of execution of the disinfection, the name of the barber or hair designer in charge, and the name of the customer having received the shaving work with the actual numerical values or the actual name printed thereon, and to develop this preparation system is equal to develop the information processing system.
Therefore, “III1. To develop a shaver disinfection contents report column preparation system” means to execute the system development, to prepare the shaver disinfection contents report column from the information processing environment.

Problem III1(1). Preparation of the shaver disinfection contents report column

The shaver disinfection contents report column is prepared after the haircut is finished by the barber by printing the items of the disinfection contents described in the paragraph of Problem III1 using the personal computer and the printer.

Problem III2. To develop a consensual validation recording column for presence or absence of shaving wounds preparation system

The barber or the hair designer has to check whether the customer gets a shaving wound or not together with the customer who has received the shaving work, fills the result in the consensual validation recording column for presence or absence of shaving wounds, and prepares the shaver disinfection contents report together with the shaver disinfection contents report column.
The shaver disinfection contents report column is a column in which the data on the shaving disinfection are printed therein by the printer, and the consensual validation recording column for presence or absence of shaving wounds is a column to be prepared by manually writing after the consensual validation of the following items relating on the shaving wound between the customer and the barber.

III2(1). Filling of the consensual validation recording column for presence or absence of shaving wounds and signing

The shaver disinfection contents report includes the consensual validation recording column for presence or absence of shaving wounds, and the customer fills either a wounded column or no wound column with the state of the wound (the place of the wound, the degree of the wound, the length of the wound xx mm, etc.) clearly, signs, and hands it to the barber.

The barber also signs on this report after receipt of the signature of the customer, prints one original and one copy of “the shaver disinfection contents report including the shaver disinfection contents report column and the consensual validation recording column for presence or absence of shaving wounds”, and hands the original to the customer, and keeps the copy for him/herself.

The barber is also required to store the contents of the shaver disinfection contents report in the form of electromagnetic information for a determined term. The shaver disinfection contents report is not limited to that printed on a piece of paper as described above, and a system to input data in an IC card and hand the card to the customer may be employed without problem.
This is a duty for both the barber and the customer for preventing epidemic of the intractable diseases.

“Advantageous effects of Invention Number III. Shaver Disinfection Contents Report Issuing System in relevant to the related art”

The Invention Number III is a system for issuing the documentary evidence proving that the shaver disinfection on individual customer basis designated by law was reliably executed, and this technology, which does not exist in the related art, serves as a foundation of demonstration of the effects described in Background Art III, IV, and V.

The “Significance of the shaver disinfection contents report” at the outset of the corresponding paragraph is also to be referred to.

“Best Mode for Carrying Out Invention Number III. Shaver Disinfection Contents Report Issuing System”

Carrying out in combination with the radiating shaver disinfector which is configured to achieve the sterilization in a short time described at the outset (reference) in DISCLOSURE OF INVENTION is the best mode.

A recommended method of recovering the cost in association with the issue of the shaver disinfection contents report is to (1) between the barber shop and the customer, charge this cost on the individual shaver disinfection fee, and (2) between the barber shop and a provider of the shaver disinfection contents report issuing system, count the number of issued shaver disinfection contents report for a certain fixed period, and the barber sends the total amount of fee corresponding thereto by bank transfer.

“Embodiment of Invention Number III. Shaver Disinfection Contents Report Issuing System”

Embodiment is now in the course of preparation and hence is not given here.

“Disclosure of Invention number IV. Development of Charging Shaver Disinfection System”

Confirmation, inspection, and discussion performed in order to solve this problem will be as follows.

IV1. Significance of Charging Shaver Disinfection System

This is a business method system for recovering equipment fund for developing and managing the shaver disinfection system on individual customer basis or additional charge for increase in working time of the barber.
It is also a scheme to intercept “an evasion saying that I cannot buy equipment for preventing the intractable diseases because I do not have enough money therefor”.

IV2. Configuration of Charging Shaver Disinfection System

A business method of handing the shaver disinfection contents report attached on a receipt on which the amount of the shaver disinfection fee is printed to the customer and in exchange for, receiving the shaver disinfection fee from the customer is defined as the charging shaver disinfection system.

The method defined here may be a business method of handing the shaver disinfection contents report attached on the receipt indicating the amounts of both the haircut fee or the beauty treatment fee and the shaver disinfection fee to the customer and in exchange for, receiving the haircut fee or the beauty treatment fee and the shaver disinfection fee from the customer or, alternatively, may be a business method of handing the shaver disinfection contents report attached to separate receipts for the haircut fee or the beauty treatment fee and the shaver disinfection fee, in exchange for, receiving the haircut fee or the beauty treatment fee and the shaver disinfection fee from the customer.

Also, the category of the shaver disinfection contents report also includes a voucher for recording and proving the contents of the shaver disinfection by means other than the heating shaver disinfector. This is for providing an occasion of issuing the lawsuit equally relating to the shaver disinfector to every customer.

The shaver disinfection contents report in Means III is “a voucher having both the shaver disinfection contents report column and the consensual validation recording column for presence or absence of shaving wounds”. However, depending on the cases, “a voucher having only the contents to be written in the shaver disinfection contents report column and having no consensual validation recording column for presence or absence of shaving wounds” is also included.

The reason is that since the present invention provides detailed means for providing the charging shaver disinfection contents report, it seems that both the customer and the barber feel easy to recover the cost, but a custom of the consensual validation for presence or absence of shaving wounds is not the rule at the moment of the invention of the present application, that is, as of Nov. 5, 2007, and the invention of the present application is lack of the detailed means for generalizing this custom.

Since the shaver disinfection contents report which describes and proves the contents of the shaver disinfection is inevitable in the site of lawsuit as a material for determining whether the shaver disinfection of a quality corresponding to the shaver disinfection fee is performed both for the heating shaver disinfector or the shaver disinfector on the basis of other principles, the customer preferably receives and keeps the shaver disinfection contents report.

IV3. Appellation and Function of Forms in Charging Shaver Disinfection System

Here, the bill, the receipt, the invoice, the shaver disinfection contents report, and the shaver disinfection contents certificate are reviewed.
When a product is delivered, the procedure of asking for the payment of the expense is, in general, in the order of:
(a)(1) submit the bill together with the invoice, (2) receive the expense, and (3) submit the receipt.
However, when the cost of the product is low and the number of transactions is high in the course of day-to-day activities, the office procedure is simplified, and
(b)(1) submit the receipt together with the invoice, (2) receive the expense, or
(c) submit the receipt (2) together with the invoice in exchange for (1) the receipt of the expense, in many cases. The deliverer has no problem even though he or she does not submit the bill as long as the expense is paid, and the purchaser will be satisfied if he or she gets the invoice and the product as written on the invoice.

The haircut fee and the shaver disinfection fee are both a small amount, and hence the form of (c) without the invoice is employed in this case according to the current custom of the payment of the haircut fee in the shop. However, since the shaver disinfection contents report which proves whether the shaver is disinfected or not has an extremely important significant as described in IV2, the customer should receive the shaver disinfection contents report positively in exchange for the shaver disinfection fee.

I would say that the shaver disinfection contents report corresponds to the invoice if being asked.
In the invention of the present application, the appellation is the shaver disinfection contents report. Even the term “a contents report”, it is used in the same meaning and contents as calling the same as the shaver disinfection contents report. In other words, the shaver disinfection contents report is provided with a function as the shaver disinfection contents certificate. Therefore, the term shaver disinfection contents certificate is not used.

“Advantageous Effects of Invention Number IV. Charging Shaver Disinfection System in relevant to Related Art” Recovery of the equipment fund of the barber shop and recovery of the expense of the shaver disinfection work are achieved without difficulty.

After having recovered the equipment fund, it becomes a source of benefit of the barber shop in the future.

“Best Mode for Carrying Out Invention Number IV. Charging Shaver Disinfection System”

Carrying out in combination with the radiating shaver disinfector which is configured to achieve the sterilization in a short time described at the outset (reference) in DISCLOSURE OF INVENTION is the best mode.

“Embodiment of Invention Number IV. Charging Shaver Disinfection System”

Embodiment is now in the course of preparation and hence is not given here.

“Disclosure of Invention Number V. Development of Insurance System against Shaving Wounds”

Confirmation, inspection, and discussion performed in order to solve this problem will be as follows.

Problem V1. Development of insurance system against shaving wounds caused by disinfected shaver

A shaving wound is often made as an actual problem. However, it is resolved by negotiation between parties in charge or is left untouched. In the barber shops which use the shaver disinfector which is able to sterilizing virus as presented in the present application, even when the shaving wound occurs, as it is “simple shaving wound which is irrelevant to the intractable disease”, the barber shops can continue business in piece by developing a small sum insurance system against the shaving wound.

Such system that the customer injured by the shaver has right to ask for the procedure for receiving the treatment on the basis of the insurance system against shaving wounds by presenting or submitting the shaver disinfection contents report to the barber is institutionalized.

In order to implement this Background Art V, it is necessary to develop Means III1 shown below, which is already developed in the paragraph III.

Means III1. A system to issue a certificate proving that the virus is sterilized (=shaver disinfection contents report) on individual custom basis.

“Advantageous Effects of Invention Number V. Insurance System against Shaving Wounds in relevant to Related Art” In the related art, the insurance system against shaving wounds could not develop by all means.

Since it is the insurance against the shaving wound for “the shaving wounds described in the consensual validation recording column for presence or absence of shaving wounds in the shaver disinfection contents report”, the intractable diseases such as AIDS or hepatitis are excluded.

“Best Mode for Carrying Out Invention Number V. Insurance System against Shaving Wounds”

Carrying out in combination with the radiating shaver disinfector which is configured to achieve the sterilization in a short time described at the outset (reference) in DISCLOSURE OF INVENTION is the best mode. A recommended method of recovering the cost in association with the operation of the insurance system against shaving wounds is to (1) between the barber shop and the customer, charge this cost on the individual shaver disinfection fee, and (2) between the barber shop and a provider of the insurance system against shaving wounds, count the number of times of the shaver disinfection for a certain fixed period and the barber sends the total amount of fee corresponding thereto by bank transfer.

“Embodiment of Invention Number V. Insurance System against Shaving Wounds”

Embodiment is now in the course of preparation and hence is not given here.

### BRIEF DESCRIPTION OF THE DRAWINGS

FIG. 1 is a plan view;

FIG. 2 is a cross-sectional view;

FIG. 3 is a perspective view; and

FIG. 4 is a cross-sectional view;

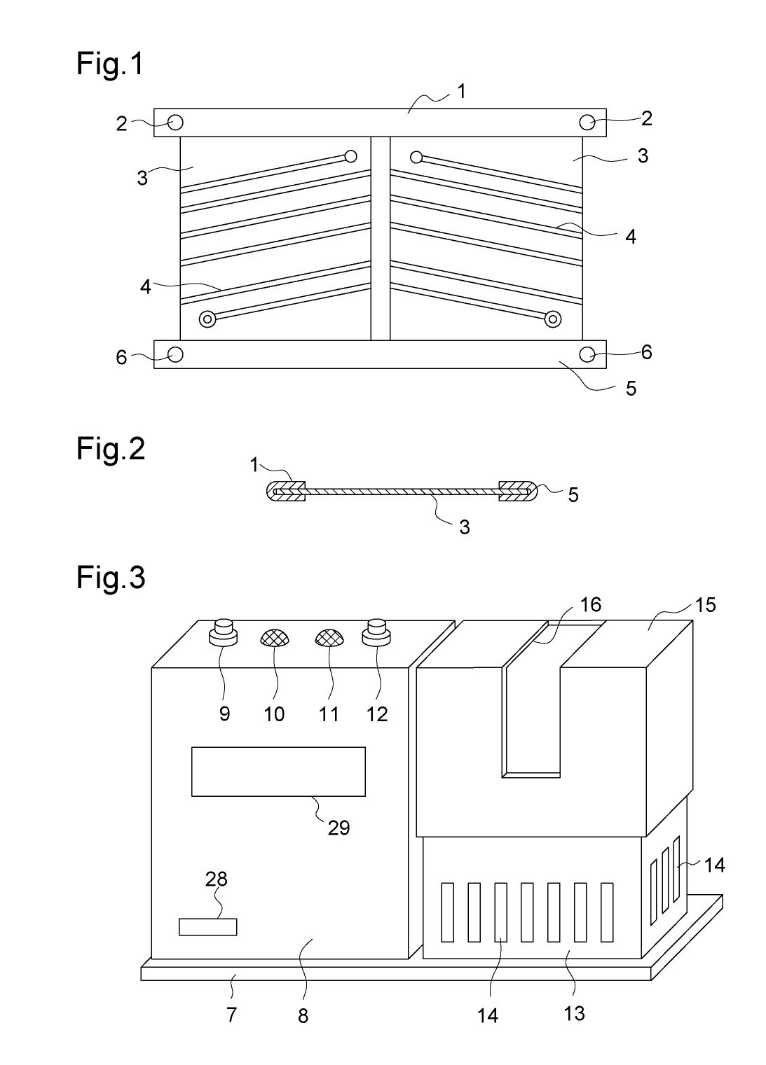

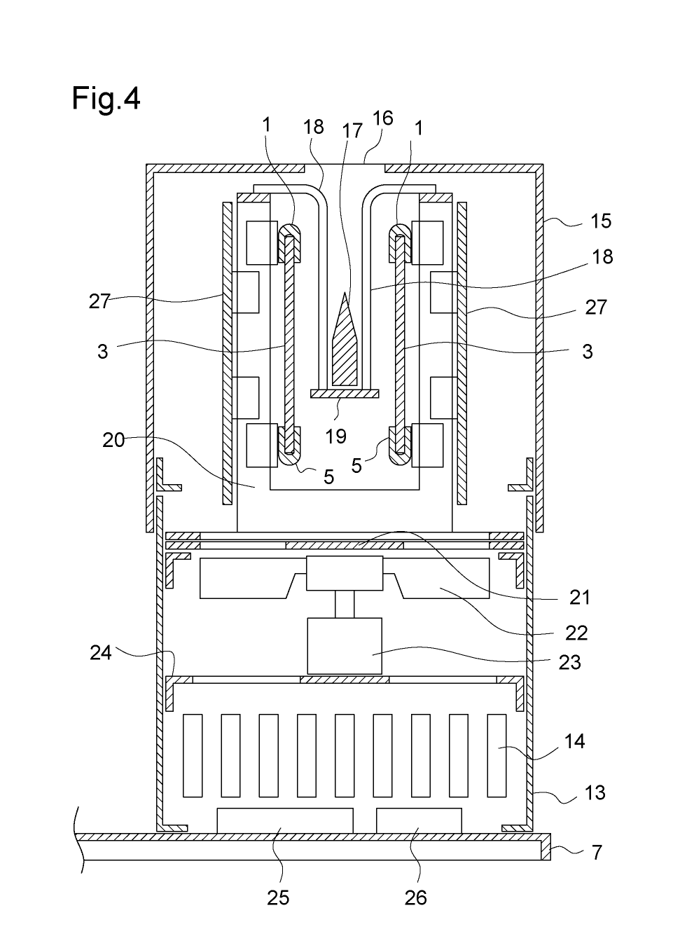

### EXPLANATIONS OF LETTERS OR NUMERALS

1 mica plate assembly fitting (upper side)
2 mica plate mounting hole (upper side)
3 mica plate
4 heating element ribbon
5 mica plate assembly fitting (lower side)
6 mica plate mounting hole (lower side)
7 base plate
8 housing for control panel, push-button, confirmation lamp system
9 black push-button switch (for emergency stop)
10 green confirmation lamp (displayed during cooling)
11 red confirmation lamp (displayed during heating)
12 red push-button switch (start of heating and start of cooling automatically when heating is completed
13 fan housing
14 air supply hole
15 heat-retention cover
16 heat-retention cover opening
17 shaver
18 shaver holder
19 shaver holder bottom plate
20 frame
21 heat-resistant partitioning plate (for protecting principal portion of a fan)
22 fan
23 fan driving motor
24 fan attachment plate
25 terminal base
26 temperature switch
27 heat reflection panel
28 electric signal acquiring and receiving terminal base or USB port
29 liquid crystal temperature display

### INDUSTRIAL APPLICABILITY

The effect on the HIV of the shaver sterilization with the experimental machine is already confirmed by specialists at our request, but it may be applied to hepatitis B, hepatitis C, and so on.

If sterilization conditions for various types of virus having shells are revealed, the heating conditions (temperature, time) will be alleviated and, in the near future, a shaver disinfection method which heats only for a period from 2 minutes to 5 minutes using the radiating shaver disinfector will be realized.
Although there is no method to express the shaver disinfection quality quantitatively so far, since the present inventor founds the method of measuring the electric signal of the respective parts of the shaver disinfector and converting the same into the temperature on individual customer basis, several new business come into the world.
The several business systems claimed in the present invention are inevitable for implementing and development of the present invention by themselves, and I believe that the range of potential users will be broadened correlatively.

1. A shaver disinfector-cum-shaver disinfection contents report issuing apparatus comprising following mechanisms from 1 to 5, and executing subsequent effects (1) and (2):
1. a mechanism for disinfecting shavers by heating the same one by one;
2. a mechanism for controlling a disinfection temperature and a disinfection time;
3. a mechanism for explaining and displaying contents of the shaver disinfection in a customer's sight;
4. a mechanism for collecting and accumulating shaver disinfection temperature data; and
5. a mechanism for issuing a shaver disinfection contents report,
(1) disinfecting the shaver by heating,
(2) issuing a shaver disinfected certificate (shaver disinfection contents report) used for a customer on individual customer basis immediately after the disinfection; wherein
the shaver disinfection contents report is made up of shaver disinfection contents report on individual customer basis including a shaver disinfection contents report column on individual customer basis having required data or items selected from recording data on heating and energization of a shaver disinfector on individual customer basis, data on the temperature in a heating chamber, data on a water temperature in a heating trough, and data on a shaver temperature, data on the length of the disinfection time, the date, month, and year of execution of the disinfection, the time of execution of the disinfection, the name of a barber or a hair designer in charge, and the name of a customer having received a shaving work with actual numerical values or the actual name printed thereon, and a consensual validation recording column for presence or absence of shaving wounds having a result of inspection about presence or absence of the shaving wound performed by the barber or the hair designer in charge in cooperation with the customer having received the shaving work.

2. A charging shaver disinfection system comprising printing a shaver disinfection contents report on individual customer basis using a sheet of paper having items printed on a back side thereof in advance using a shaver disinfector-cum-shaver disinfection contents report issuing apparatus according to claim 1, handing the same to a customer, and receiving a shaver disinfection fee from the customer, the items to be printed on the back side of the sheet including:
1. an explanation of the charging shaver disinfection system;
2. agreement and request statement of the customer saying that the customer agrees with the system and makes a request for the shaver disinfection;
3. name of the customer (blank but to be signed by the customer himself or herself after having agreed with the system); and
4. a name of the barber shop.

3. An insurance system against shaving wounds comprising printing a shaver disinfection contents report on individual customer basis using a shaver disinfector-cum-shaver disinfection contents report issuing apparatus according to claim 1, handing the shaver disinfection contents report attached with;
1. a certificate of consent including a sentence saying that a customer agrees with general contractual conditions of the insurance against shaving wounds and agrees with being insured and a signature column (blank) for the customer printed thereon;
2. general contractual conditions of the insurance against shaving wounds including a name of the barber shop printed thereon;
to the customer and obtaining the customer's signature in the signature column to enter into an agreement for insurance and collecting an expense for the insurance against shaving wounds, or
an insurance system against shaving wounds including
printing a shaver disinfection contents report on individual customer basis using a sheet of paper having items printed on a back side of the sheet instead of “handing the shaver disinfection contents report attached with; 1. a certificate of consent including a sentence saying that a customer agrees with general contractual conditions of the insurance against shaving wounds and agrees with being insured and a signature column (blank) printed thereon; 2. the general contractual conditions of the insurance against shaving wounds including a name of the barber shop printed thereon; to the customer”, handing the same to the customer, obtaining a signature in a signature column, and collecting an expense for the insurance against shaving wounds, the items including;
1. the general contractual conditions of the insurance against shaving wounds
2. the signature column (blank) for the customer to be filled when the customer agrees with the general contractual conditions of the insurance against shaving wounds and agrees with being insured
3. a name of the barber shop.
